(19) United States
(12) Patent Application Publication
(10) Pub. No.: US 2021/0238177 A1
(43) Pub. Date: Aug. 5, 2021

(54) CRYSTALLINE FORMS OF 3-((1R,5S,9r)-9-ETHOXY-3-AZABICYCLO[3.3.1]NONAN-9-YL)-BENZAMIDE AND SALT FORMS THEREOF

(71) Applicant: Alkermes, Inc., Waltham, MA (US)

(72) Inventors: J. Michael MacPhee, Pawtucket, RI (US); David Robert Webster, Sudbury, MA (US); Mark David Tawa, Acton, MA (US); Lisa Ferreira, Franklin, MA (US)

(73) Assignee: Alkermes, Inc., Waltham, MA (US)

(21) Appl. No.: 17/166,340

(22) Filed: Feb. 3, 2021

Related U.S. Application Data
(60) Provisional application No. 62/969,841, filed on Feb. 4, 2020.

(51) Int. Cl. C07D 471/08

(52) U.S. Cl. CPC C07D 471/08; C07B 2200/13

(57) ABSTRACT

This disclosure provides crystalline forms of a μ receptor agonist, and methods of making and using these forms.

### RELATED APPLICATIONS

This application claims priority to U.S. Provisional Application No. 62/969,841 filed Feb. 4, 2020, the entire content of which is hereby incorporated in its entirety.

### BACKGROUND

Pain is the most common reason for physician consultation, and can be caused by a variety of medical conditions and procedures. Both chronic and acute pain can lead to a significant reduction in the quality of life, with many individuals facing long term disablement and handicaps.

Opiates have been the subject of intense research since the isolation of morphine in 1805, and thousands of compounds having opiate or opiate-like activity have been identified. Many opioid receptor-interactive compounds including those used for producing analgesia (e.g., morphine) and those used for treating drug addiction (e.g., naltrexone and cyclazocine) have been employed in human therapy. The actions of endogenous opioids and opiates are mediated by three receptor types (μ, δ, and κ receptors), which are coupled to different intracellular effector systems. [Berrocoso E. et. al., Current Pharmaceutical Design, 15(14) 2009, 1612-22]. As such, agents that can modulate the actions of one or more of the opioid receptor types with selectivity and sensitivity are important to treat the various diseases and disorders regulated by the opioid system. Compounds that bind to opioid receptors are likely to be useful in the treatment of diseases and conditions modulated by opiate receptors.

Traditional opioid analgesics exert their pharmacological activity once they have passed into the central nervous system (CNS). But this can lead to undesirable CNS-mediated side effects, such as respiratory depression, increased drug tolerance, increased drug dependence, constipation and unwanted euphoria. While certain treatments for pain do exist, many commonly used analgesics suffer from significant drawbacks including inefficacy, tolerance, and chemical dependence. There is therefore a need for new compounds and methods of treatment for pain that may be used alone or in conjunction with existing therapeutic modalities.

### SUMMARY

Provided herein are crystalline forms useful for the treatment of pain in a subject in need thereof. In an aspect, provided herein are crystalline forms of 3-(1R,5S,9r-9-ethoxy-3-azabicyclo[3.3.1]nonan-9-yl)-benzamide free base having the formula:

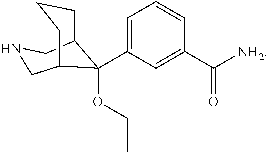 [SMILES: CCOC1(c2cccc(C(N)=O)c2)C2CCCC1CNC2]

In another aspect, provided herein is a method of treating pain in a subject in need thereof comprising administering to the subject a crystalline form of 3-((1R,5S,9r-9-ethoxy-3-azabicyclo-[3.3.1]nonan-9-yl)-benzamide free base.

In another aspect, provided herein are crystalline forms of 3-((1R,5S,9r-9-ethoxy-3-azabicyclo[3.3.1]nonan-9-yl)-benzamide hydrochloride having the formula:

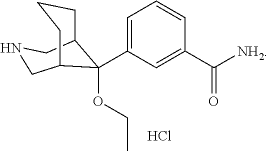 [SMILES: CCOC1(c2cccc(C(N)=O)c2)C2CCCC1CNC2 ; Cl]

In another aspect, provided herein is a method of treating pain in a subject in need thereof comprising administering to the subject a crystalline form of 3-((1R,5S,9r)-9-ethoxy-3-azabicyclo-[3.3.1]nonan-9-yl)-benzamide hydrochloride.

### BRIEF DESCRIPTION OF THE DRAWINGS

FIG. 1 shows the PXRD diffractogram of crystalline 3-((1R,5S,9r-9-ethoxy-3-azabicyclo[3.3.1]nonan-9-yl)-benzamide free base Form A.

FIG. 2 shows the DSC thermogram of crystalline 3-((1R,5S,9r-9-ethoxy-3-azabicyclo[3.3.1]nonan-9-yl)-benzamide free base Form A.

FIG. 3 shows the PXRD diffractogram of crystalline 3-((1R,5S,9r-9-ethoxy-3-azabicyclo[3.3.1]nonan-9-yl)-benzamide hydrochloride Form A.

FIG. 4 shows the DSC thermogram of crystalline 3-((1R,5S,9r-9-ethoxy-3-azabicyclo[3.3.1]nonan-9-yl)-benzamide hydrochloride Form A.

FIG. 5 shows the PXRD diffractogram of crystalline 3-((1R,5S,9r-9-ethoxy-3-azabicyclo[3.3.1]nonan-9-yl)-benzamide hydrochloride Form B.

FIG. 6 shows the DSC thermogram of crystalline 3-((1R,5S,9r-9-ethoxy-3-azabicyclo[3.3.1]nonan-9-yl)-benzamide hydrochloride Form B.

FIG. 7 shows the PXRD diffractogram of crystalline 3-((1R,5S,9r-9-ethoxy-3-azabicyclo[3.3.1]nonan-9-yl)-benzamide hydrochloride Form D.

FIG. 8 shows the DSC thermogram of crystalline 3-((1R,5S,9r-9-ethoxy-3-azabicyclo[3.3.1]nonan-9-yl)-benzamide hydrochloride Form D.

FIG. 9 shows the DVS isotherm of crystalline 3-((1R,5S,9r-9-ethoxy-3-azabicyclo[3.3.1]nonan-9-yl)-benzamide hydrochloride Form D.

### DETAILED DESCRIPTION

The solid state of a compound can be important when the compound is used for pharmaceutical purposes. The physical properties of a compound can change from one solid form to another, which can affect the suitability of the form for pharmaceutical use. For example, a particular crystalline solid compound can overcome the disadvantage of other solid forms of the compound such as, e.g., instability and/or reduced purity.

Provided herein are solid, crystalline forms of 3-((1R,5S,9r-9-ethoxy-3-azabicyclo-[3.3.1]nonan-9-yl)-benzamide. This compound is useful for the treatment of pain in a subject:

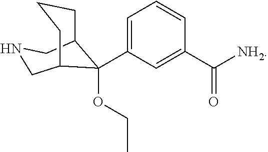 [SMILES: CCOC1(c2cccc(C(N)=O)c2)C2CCCC1CNC2]

In particular, provided herein are crystalline forms of 3-((1R,5S,9r-9-ethoxy-3-azabicyclo-[3.3.1]nonan-9-yl)-benzamide free base and crystalline forms of 3-((1R,5S,9r)-9-ethoxy-3-azabicyclo-[3.3.1]nonan-9-yl)-benzamide hydrochloride.

This compound is disclosed in PCT Publication No. WO 2019/152946 and U.S. patent application Ser. No. 16/267,025, the entire contents of which are incorporated herein by reference.

The crystalline forms provided herein can be characterized by powder X-ray diffraction (PXRD), differential scanning calorimetry (DSC), and thermogravimetric analysis (TGA).

In one embodiment, the crystalline form of the free base (form A) disclosed herein is characterized by an PXRD diffractogram having peaks expressed in degrees-2-theta at angles (±0.2 degrees) of 6.7, 9.9, and 13.6.

In another embodiment, the crystalline form of the hydrochloride salt (form A) disclosed herein is characterized by an PXRD diffractogram having peaks expressed in degrees-2-theta at angles (±0.2 degrees) of 10.9, 12.2, and 13.7.

In another embodiment, the crystalline form of the hydrochloride salt (form B) disclosed herein is characterized by an PXRD diffractogram having peaks expressed in degrees-2-theta at angles (±0.2 degrees) of 9.0, 10.8, and 12.4.

In a non-limiting aspect, this compound modulates the μ-opioid receptor. In a particular embodiment, the compound provided herein is a μ-receptor agonist. As such, in one aspect, the compound provided herein is useful in treatment of pain in a subject by acting as an agonist of the μ-receptor.

### Definitions

Listed below are definitions of various terms used to describe the crystalline forms provided herein. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art to which the compound and its crystalline forms belong. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used to herein, the term “EC₅₀” refers to the concentration of a compound required to achieve an effect that is 50% of the maximal observed effect of a compound.

The term “agonist,” as used herein, refers to a compound that, when contacted with a target of interest (e.g., the μ-opioid receptor) causes an increase in the magnitude of a certain activity or function of the target compared to the magnitude of the activity or function observed in the absence of the agonist.

As used herein, “pain” is generally defined as physical suffering or discomfort caused by illness or injury, and can be thought of as encompassing inflammatory pain, thermal pain, acute pain, chronic pain, musculoskeletal pain, post-surgical pain, nociceptive pain, neuropathic pain, and the like.

As used herein, the term “depression” can be generally defined as a mental condition characterized by feelings of severe despondency and dejection. “Depression” can also be referred to as major depression, clinical depression, major depressive illness, major affective disorder and unipolar mood disorder. The depressive condition can be an anxiety disorder, a mental condition, recurrent depression, and the like.

As used herein, addiction is generally defined as a chronic brain disease that causes compulsive drug seeking and use, or alcohol seeking and use. Drug addiction can be opioid addiction (i.e., opioid dependence), stimulant addiction, and the like.

As used herein, the term “pharmaceutically acceptable carrier” refers to a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the present disclosure within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound provided herein, and not injurious to the patient.

Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of the compound provided herein, and are physiologically acceptable to the patient. Supplementary active compounds may also be incorporated into the compositions. Other additional ingredients that may be included in the pharmaceutical compositions used in the practice of the present disclosure are known in the art and described, for example in Remington's Pharmaceutical Sciences (Genaro, Ed., Mack Publishing Co., 1985, Easton, Pa.), which is incorporated herein by reference.

As used herein, the phrases “therapeutically effective dose” and “therapeutically effective amount” refer to an amount of a compound that prevents the onset, alleviates the symptoms, stops the progression of a disease, or results in another desired biological outcome such as, e.g., improved clinical signs.

The term “treat,” “treated,” “treating,” or “treatment” includes the diminishment or alleviation of at least one symptom associated or caused by the state, disorder or disease being treated. In certain embodiments, the treatment comprises bringing into contact with the opioid receptor an effective amount of the compound provided herein for conditions related to pain, depression or addiction.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “patient,” “individual” or “subject” refers to a human or a non-human mammal. Non-human mammals include, for example, livestock and pets, such as ovine, bovine, porcine, canine, feline and murine mammals. In an embodiment, the patient, subject, or individual is human.

The term “administering” or “administration” and the like, refers to providing a therapeutic agent, such as a crystalline form disclosed herein, to the subject in need of treatment. In an embodiment, the subject is a mammal. In another embodiment, the subject is a human.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, including ±5%, ±1%, and ±0.1% from the specified value, as such variations are appropriate to perform the disclosed methods.

### Characterization of Crystalline Forms

In certain embodiments, the crystalline forms described herein are identifiable on the basis of characteristic peaks in a powder X-ray diffraction analysis. Powder X-ray diffraction (PXRD) is a scientific technique using X-ray, neutron, or electron diffraction on powder, microcrystalline, or other solid materials for structural characterization of solid materials. A description of the methods used to obtain certain PXRD diffractograms in connection with the crystalline forms provided herein can be found in the Examples below. In an embodiment, the X-ray powder diffraction data provided herein is obtained by a method utilizing Cu Kα radiation.

3-((1R,5S,9r-9-ethoxy-3-azabicyclo-[3.3.1]nonan-9-yl)-benzamide Free Base Form A

In an aspect, provided herein is the crystalline form 3-((1R,5S,9r-9-ethoxy-3-azabicyclo-[3.3.1]nonan-9-yl)-benzamide free base Form A.

In another embodiment, the crystalline form of the free base Form A is characterized by an PXRD diffractogram having peaks expressed in degrees-2-theta at angles (±0.2 degrees) of 6.7, 9.9 and 13.6.

In another embodiment, the crystalline form of the free base Form A is characterized by an PXRD diffractogram having peaks expressed in degrees-2-theta at angles (±0.2 degrees) of 6.7, 11.4 and 12.2.

In another embodiment, the crystalline form of the free base Form A is characterized by an PXRD diffractogram having peaks expressed in degrees-2-theta at angles (±0.2 degrees) of 9.9, 15.1 and 18.4.

In another embodiment, the crystalline form of the free base Form A is characterized by an PXRD diffractogram having peaks expressed in degrees-2-theta at angles (±0.2 degrees) of 9.9, 11.4, 13.6 and 15.1.

In another embodiment, the crystalline form of the free base Form A is characterized by an PXRD diffractogram having peaks expressed in degrees-2-theta at angles (±0.2 degrees) of: 6.7, 9.9, 12.2 and 15.1.

In another embodiment, the crystalline form of the free base Form A is characterized by an PXRD diffractogram having peaks expressed in degrees-2-theta at angles (±0.2 degrees) of: 6.7, 9.9, 11.4, 12.2, 13.6 and 15.1.

In another embodiment, the crystalline form of the free base Form A is characterized by an PXRD diffractogram having peaks expressed in degrees-2-theta at angles (±0.2 degrees) of: 6.7, 9.9, 11.4, 12.2, 13.6, 15.1, 18.4, 20.3 and 20.6.

In another embodiment, the crystalline form of the free base Form A is characterized by an PXRD diffractogram having peaks expressed in degrees-2-theta at angles (±0.2 degrees) of:

Free Base Form A

| Peak Number | Angle (degrees 2θ) |
|---|---|
| 1 | 6.7 |
| 2 | 9.9 |
| 3 | 11.4 |
| 4 | 12.2 |
| 5 | 12.6 |
| 6 | 13.6 |
| 7 | 14.3 |
| 8 | 14.5 |
| 9 | 15.1 |
| 10 | 15.6 |
| 11 | 17.2 |
| 12 | 18.4 |
| 13 | 19.2 |
| 14 | 20.3 |
| 15 | 20.6 |
| 16 | 22.6 |
| 17 | 23.1 |
| 18 | 24.2 |
| 19 | 25.6 |
| 20 | 26.2 |
| 21 | 27.0 |
| 22 | 27.6 |
| 23 | 28.4 |
| 24 | 31.5 |

In another embodiment, the crystalline form of the free base Form A is characterized by any two, three, four, five, six, seven, or eight peaks from the list above.

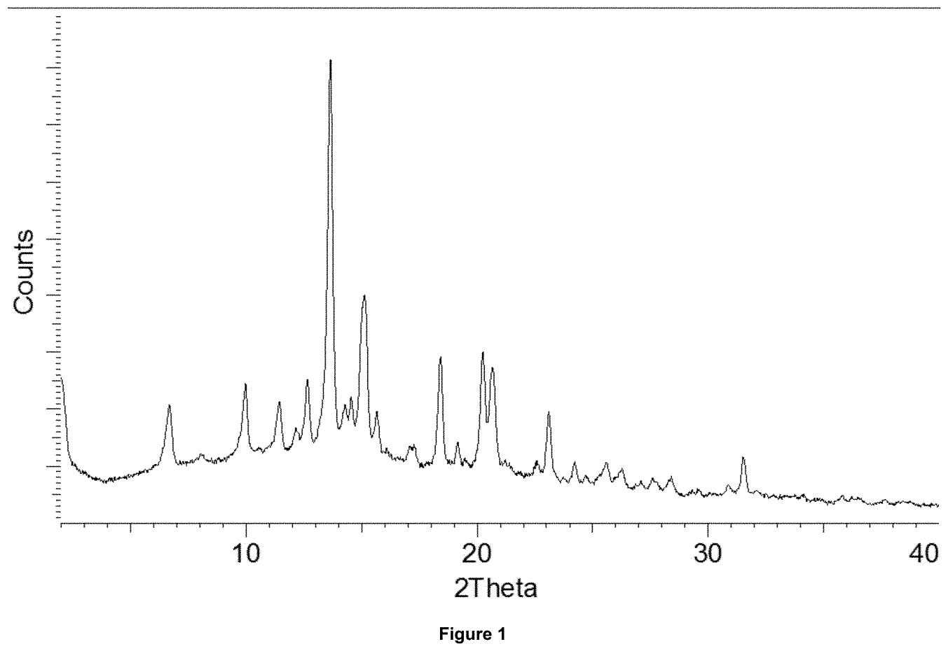

In another embodiment, the crystalline form of the free base Form A has an PXRD diffractogram substantially as depicted in FIG. 1.

In another embodiment, the crystalline form of the free base Form A has a DSC thermogram characterized by an endotherm with an onset temperature of 176 degrees C.

3-((1R,5S,9r-9-ethoxy-3-azabicyclo-[3.3.1]nonan-9-yl)-benzamide Hydrochloride Form A

In another aspect, provided herein is the crystalline form 3-((1R,5S,9r-9-ethoxy-3-azabicyclo-[3.3.1]nonan-9-yl)-benzamide hydrochloride Form A.

In another embodiment, the crystalline form of the hydrochloride Form A is characterized by an PXRD diffractogram having peaks expressed in degrees-2-theta at angles (±0.2 degrees) of 10.9, 12.2 and 13.7.

In another embodiment, the crystalline form of the hydrochloride Form A is characterized by an PXRD diffractogram having peaks expressed in degrees-2-theta at angles (±0.2 degrees) of 12.2, 12.9 and 15.5.

In another embodiment, the crystalline form of the hydrochloride Form A is characterized by an PXRD diffractogram having peaks expressed in degrees-2-theta at angles (±0.2 degrees) of 12.2, 17.2 and 18.4.

In another embodiment, the crystalline form of the hydrochloride Form A is characterized by an PXRD diffractogram having peaks expressed in degrees-2-theta at angles (±0.2 degrees) of 10.9, 12.2, 13.7 and 15.5.

In another embodiment, the crystalline form of the hydrochloride Form A is characterized by an PXRD diffractogram having peaks expressed in degrees-2-theta at angles (±0.2 degrees) of 10.9, 12.2, 12.9, 13.7 and 15.5.

In another embodiment, the crystalline form of the hydrochloride Form A is characterized by an PXRD diffractogram having peaks expressed in degrees-2-theta at angles (±0.2 degrees) of 10.9, 12.2, 12.9, 13.7, 15.5, 17.2 and 18.4.

In another embodiment, the crystalline form of the hydrochloride Form A is characterized by an PXRD diffractogram having peaks expressed in degrees-2-theta at angles (±0.2 degrees) of 10.9, 12.2, 12.9, 13.7, 15.5, 17.2, 18.4, and 19.9.

In another embodiment, the crystalline form of the hydrochloride Form A is characterized by an PXRD diffractogram having peaks expressed in degrees-2-theta at angles (±0.2 degrees) of:

Hydrochloride Form A

| Peak Number | Angle (degrees 2θ) |
|---|---|
| 1 | 10.9 |
| 2 | 12.2 |
| 3 | 12.9 |
| 4 | 13.7 |
| 5 | 15.5 |
| 6 | 17.2 |
| 7 | 18.4 |
| 8 | 19.9 |
| 9 | 21.0 |
| 10 | 22.1 |
| 11 | 24.5 |
| 12 | 26.3 |
| 13 | 26.7 |
| 14 | 27.2 |
| 15 | 27.8 |
| 16 | 29.1 |
| 17 | 29.8 |
| 18 | 30.7 |
| 19 | 31.5 |

In another embodiment, the crystalline form of the hydrochloride Form A is characterized by any two, three, four, five, six, seven, or eight peaks from the list above.

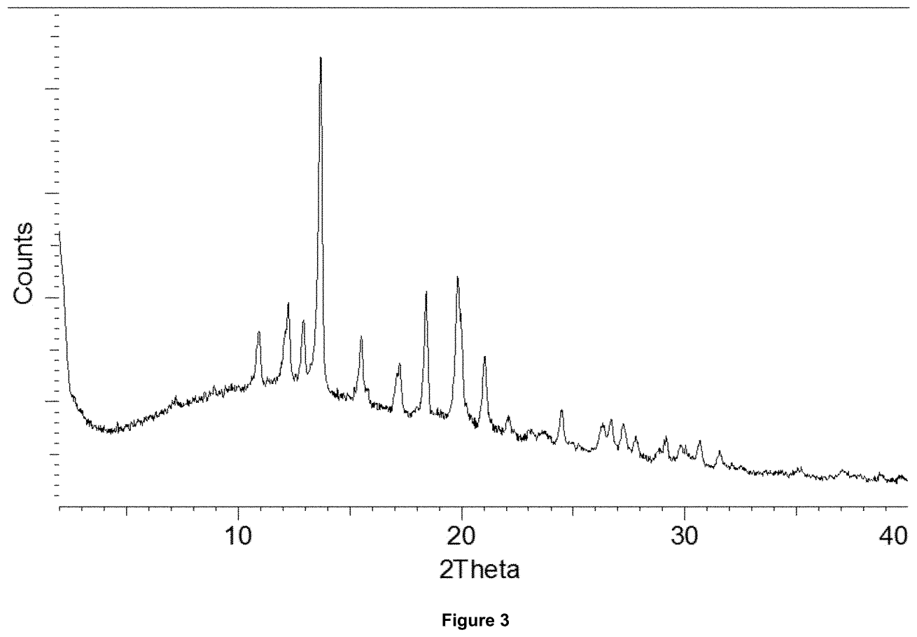

In another embodiment, the crystalline form of the hydrochloride Form A has an PXRD diffractogram substantially as depicted in FIG. 3.

In another embodiment, the crystalline form of the hydrochloride Form A has a DSC thermogram characterized by an endotherm with an onset temperature of 164 degrees C.

3-((1R,5S,9r-9-ethoxy-3-azabicyclo-[3.3.1]nonan-9-yl)-benzamide hydrochloride Form B

In another aspect, provided herein is the crystalline form 3-((1R,5S,9r-9-ethoxy-3-azabicyclo-[3.3.1]nonan-9-yl)-benzamide hydrochloride Form B.

In another embodiment, the crystalline form of the hydrochloride Form B is characterized by an PXRD diffractogram having peaks expressed in degrees-2-theta at angles (±0.2 degrees) of 10.8, 14.7, and 15.4.

In another embodiment, the crystalline form of the hydrochloride Form B is characterized by an PXRD diffractogram having peaks expressed in degrees-2-theta at angles (±0.2 degrees) of 9.0, 10.0, and 10.8.

In another embodiment, the crystalline form of the hydrochloride Form B is characterized by an PXRD diffractogram having peaks expressed in degrees-2-theta at angles (±0.2 degrees) of 9.0, 10.8, and 12.4.

In another embodiment, the crystalline form of the hydrochloride Form B is characterized by an PXRD diffractogram having peaks expressed in degrees-2-theta at angles (±0.2 degrees) of 9.0, 10.8, and 15.4.

In another embodiment, the crystalline form of the hydrochloride Form B is characterized by an PXRD diffractogram having peaks expressed in degrees-2-theta at angles (±0.2 degrees) of 9.0, 10.0, 10.8 and 12.4.

In another embodiment, the crystalline form of the hydrochloride Form B is characterized by an PXRD diffractogram having peaks expressed in degrees-2-theta at angles (±0.2 degrees) of 9.0, 10.0, 10.8, 12.4 and 14.7.

In another embodiment, the crystalline form of the hydrochloride Form B is characterized by an PXRD diffractogram having peaks expressed in degrees-2-theta at angles (±0.2 degrees) of 9.0, 10.0, 10.8, 12.4 and 15.4.

In another embodiment, the crystalline form of the hydrochloride Form B is characterized by an PXRD diffractogram having peaks expressed in degrees-2-theta at angles (±0.2 degrees) of 9.0, 10.8, 12.4, and 14.7.

In another embodiment, the crystalline form of the hydrochloride Form B is characterized by an PXRD diffractogram having peaks expressed in degrees-2-theta at angles (±0.2 degrees) of 9.0, 10.8, 14.7, and 15.4.

In another embodiment, the crystalline form of the hydrochloride Form B is characterized by an PXRD diffractogram having peaks expressed in degrees-2-theta at angles (±0.2 degrees) of 9.0, 10.8, 12.4, 14.7, 15.4, 18.5 and 20.1.

In another embodiment, the crystalline form of the hydrochloride Form B is characterized by an PXRD diffractogram having peaks expressed in degrees-2-theta at angles (±0.2 degrees) of 9.0, 10.8, 14.7, 15.4, 18.5, 20.1, and 22.3.

In another embodiment, the crystalline form of the hydrochloride Form B is characterized by an PXRD diffractogram having peaks expressed in degrees-2-theta at angles (±0.2 degrees) of 9.0, 10.0, 10.8, 12.4, 14.7, 15.4, 18.1, 18.5, 20.1, and 22.3.

In another embodiment, the crystalline form of the hydrochloride Form B is characterized by an PXRD diffractogram having peaks expressed in degrees-2-theta at angles (±0.2 degrees) of:

Hydrochloride Form B

| Peak Number | Angle (degrees 2θ) |
|---|---|
| 1 | 9.0 |
| 2 | 10.0 |
| 3 | 10.8 |
| 4 | 12.4 |
| 5 | 12.8 |
| 6 | 14.7 |
| 7 | 15.4 |
| 8 | 16.6 |
| 9 | 17.6 |
| 10 | 18.1 |
| 11 | 18.5 |
| 12 | 19.3 |
| 13 | 20.1 |
| 14 | 20.3 |
| 15 | 21.7 |
| 16 | 22.0 |
| 17 | 22.3 |
| 18 | 23.2 |
| 19 | 24.4 |
| 20 | 25.1 |
| 21 | 25.3 |
| 22 | 25.9 |
| 23 | 26.2 |
| 24 | 27.5 |
| 25 | 28.5 |
| 26 | 29.8 |
| 27 | 30.6 |
| 28 | 32.0 |
| 29 | 33.1 |
| 30 | 33.7 |
| 31 | 34.5 |
| 32 | 35.1 |
| 33 | 35.5 |
| 34 | 39.2 |

In another embodiment, the crystalline form of the hydrochloride Form B is characterized by any two, three, four, five, six, seven, or eight peaks from the list above.

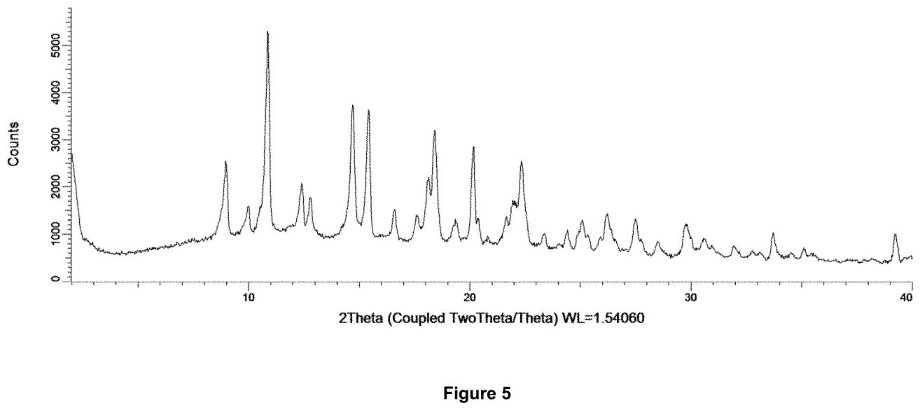

In another embodiment, the crystalline form of the hydrochloride Form B has the PXRD diffractogram substantially as depicted in FIG. 5.

In another embodiment, the crystalline form of the hydrochloride Form B has a DSC thermogram characterized by an endotherm with an onset temperature of 264 degrees C.

3-((1R,5S,9r-9-ethoxy-3-azabicyclo-[3.3.1]nonan-9-yl)-benzamide Hydrochloride Form D

In another aspect, provided herein is the crystalline form 3-((1R,5S,9r-9-ethoxy-3-azabicyclo-[3.3.1]nonan-9-yl)-benzamide hydrochloride Form D.

In another embodiment, the crystalline form of the hydrochloride Form D is characterized by an PXRD diffractogram having peaks expressed in degrees-2-theta at angles (±0.2 degrees) of 9.1, 10.4, and 13.8.

In another embodiment, the crystalline form of the hydrochloride Form D is characterized by an PXRD diffractogram having peaks expressed in degrees-2-theta at angles (±0.2 degrees) of 9.1, 10.4, 11.9, and 13.8.

In another embodiment, the crystalline form of the hydrochloride Form D is characterized by an PXRD diffractogram having peaks expressed in degrees-2-theta at angles (±0.2 degrees) of 9.1, 10.4, 11.9, 13.8, and 15.8.

In another embodiment, the crystalline form of the hydrochloride Form D is characterized by an PXRD diffractogram having peaks expressed in degrees-2-theta at angles (±0.2 degrees) of 9.1, 10.4, 11.9, 13.8, 15.8, and 19.6.

In another embodiment, the crystalline form of the hydrochloride Form D is characterized by an PXRD diffractogram having peaks expressed in degrees-2-theta at angles (±0.2 degrees) of 9.1, 10.4, 11.9, 13.8, 15.8, 19.6, and 21.6.

In another embodiment, the crystalline form of the hydrochloride Form D is characterized by an PXRD diffractogram having peaks expressed in degrees-2-theta at angles (±0.2 degrees) of 9.1, 10.4, 11.6, 11.9, 13.8, 15.8, 17.6, 19.6, and 21.6.

In another embodiment, the crystalline form of the hydrochloride Form D is characterized by an PXRD diffractogram having peaks expressed in degrees-2-theta at angles (±0.2 degrees) of:

Hydrochloride Form D

| Peak Number | Angle (degrees 2θ) |
|---|---|
| 1 | 9.1 |
| 2 | 9.7 |
| 3 | 10.4 |
| 4 | 11.6 |
| 5 | 11.9 |
| 6 | 13.8 |
| 7 | 15.8 |
| 8 | 17.1 |
| 9 | 17.6 |
| 10 | 18.3 |
| 11 | 19.0 |
| 12 | 19.6 |
| 13 | 21.0 |
| 14 | 21.6 |
| 15 | 21.7 |
| 16 | 22.5 |
| 17 | 23.9 |
| 18 | 24.1 |
| 19 | 25.1 |
| 20 | 26.1 |
| 21 | 27.6 |
| 22 | 29.2 |
| 23 | 29.5 |
| 24 | 30.4 |
| 25 | 30.7 |
| 26 | 31.7 |
| 27 | 32.1 |
| 28 | 32.8 |
| 29 | 33.3 |
| 30 | 33.7 |
| 31 | 34.2 |
| 32 | 34.9 |
| 33 | 36.6 |
| 34 | 38.2 |

In another embodiment, the crystalline form of the hydrochloride Form D is characterized by any two, three, four, five, six, seven, or eight peaks from the list above.

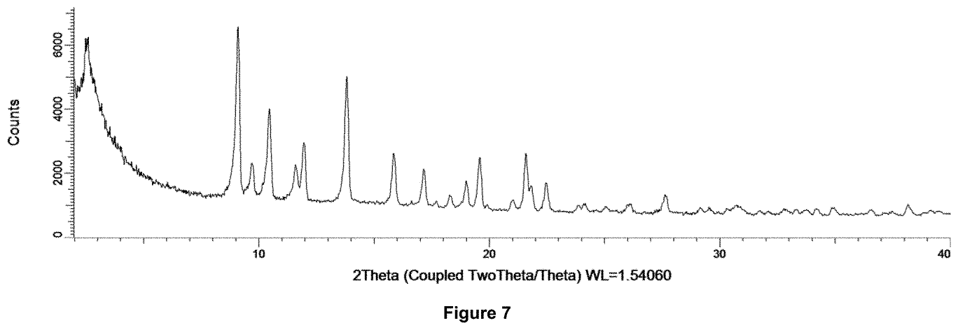

In another embodiment, the crystalline form of the hydrochloride Form D has the PXRD diffractogram substantially as depicted in FIG. 7.

In another embodiment, the crystalline form of the hydrochloride Form D has a DSC thermogram characterized by an endotherm with an onset temperature of 263 degrees C.

### Methods of Treatment

Provided herein are methods for the treatment of a disease comprising administering a crystalline form of 3-((1R,5S,9r-9-ethoxy-3-azabicyclo[3.3.1]nonan-9-yl)-benzamide, or a pharmaceutical composition comprising the crystalline form and a pharmaceutically acceptable carrier.

In an aspect, provided herein is a method of treating pain in a subject in need thereof comprising administering to the subject the crystalline form, or a pharmaceutical composition comprising the crystalline form and a pharmaceutically acceptable carrier.

In another embodiment, the pain is selected from inflammatory pain, thermal pain, acute pain, chronic pain, traumatic pain, chemical pain, ischemic pain, centrally mediated pain, peripherally mediated pain, prickling pain, visceral pain, progressive disease pain, musculoskeletal pain (e.g., back pain, neck pain), post-surgical pain, bone pain (e.g., osteoarthritis), nociceptive pain, or neuropathic pain. In another embodiment, the pain is inflammatory pain, thermal pain, acute pain, chronic pain, or neuropathic pain. In another embodiment, the pain is musculoskeletal pain (e.g., back pain, neck pain), post-surgical pain, or bone pain (e.g., osteoarthritis).

In another embodiment, the pain is inflammatory pain, thermal pain, acute pain, chronic pain, traumatic pain, chemical pain, ischemic pain, centrally mediated pain, peripherally mediated pain, prickling pain, visceral pain, progressive disease pain, musculoskeletal pain, and neuropathic pain.

In yet another embodiment, the pain can be chronic pain, wherein the pain is chronic pain from headache, chronic pain from neuropathic conditions, chronic pain from post-stroke conditions or chronic pain from migraine.

In still another embodiment, the pain can be acute pain, wherein the pain is acute pain from acute injury, acute pain from trauma, or acute pain from surgery.

In a particular embodiment, the pain is inflammatory pain, thermal pain, acute pain, chronic pain, musculoskeletal pain, and neuropathic pain. In another embodiment, the pain is chromic pain. In another embodiment, the pain is musculoskeletal pain.

In one embodiment, the pain can be neuropathic pain, wherein the pain is neuropathic pain from alcoholic polyneuropathy, phantom limb pain, chemotherapy, diabetic pain, pain from HIV infection or AIDS, multiple sclerosis, shingles, Parkinson's disease, spine surgery, or postherpetic neuralgia.

In one embodiment, the pain can be inflammatory pain, wherein the pain is pain associated with arthritis such as rheumatoid arthritis, rheumatoid spondylitis, osteoarthritis, gouty arthritis, juvenile arthritis, or scapulohumeral periarthritis.

In an embodiment, the pain is inflammatory pain, thermal pain, acute pain, chronic pain, traumatic pain, chemical pain, ischemic pain, centrally mediated pain, peripherally mediated pain, prickling pain, visceral pain, progressive disease pain, musculoskeletal pain and neuropathic pain.

In another embodiment, the pain is inflammatory pain, thermal pain, acute pain, chronic pain, musculoskeletal pain, and neuropathic pain. In yet another embodiment, the pain is chronic pain. In still another embodiment, the pain is musculoskeletal pain.

In another aspect, provided herein is a method of treating pain in a subject in need thereof comprising administering to the subject the crystalline form, or a pharmaceutical composition comprising the crystalline form and a pharmaceutically acceptable carrier, wherein said treatment refers to the diminishment or alleviation of at least one symptom associated or caused by the state, disorder or disease being treated.

In another aspect, provided herein is a method of treating pain in a subject in need thereof comprising administering to the subject the crystalline form, or a pharmaceutical composition comprising the crystalline form and a pharmaceutically acceptable carrier, wherein said treatment refers to ameliorating or inhibiting the pain in a patient.

In yet another aspect, provided herein is a method of preventing pain in a subject in need thereof comprising administering to the subject the crystalline form, or a pharmaceutical composition comprising the crystalline form and a pharmaceutically acceptable carrier.

In an aspect, the crystalline forms disclosed herein can be used to treat depression in a subject in need thereof. As used herein, the term “depression” refers to “clinical depression” or “major depressive disorder.”

In another embodiment, the crystalline forms disclosed herein can be used to treat a depressive condition in a subject in need thereof. In an embodiment, the depressive condition is depressed mood, diminished concentration, insomnia, fatigue, loss of appetite, excessive guilt, and suicidal thoughts. The depressive condition can be an anxiety disorder, wherein the anxiety disorder is generalized anxiety disorder, panic, or agoraphobia. The depressive condition can be associated with a mental condition, wherein the mental condition is schizoaffective disorder, or seasonal affective disorder. The depressive condition can be associated with chronic or recurrent depression. The depressive condition can be depressed mood, loss of pleasure, loss of appetite, sleep disturbance, psychomotor changes, fatigue, or post-partum depression. The depressive condition can be adjustment disorders with depressed mood, Asperger syndrome, attention deficit, bereavement, bipolar I disorder, bipolar II disorder, borderline and personality disorder, cyclothymia and dysthymia, Dysthymic disorder, hyperactivity disorder, impulse control disorder, mixed mania, obsessive-compulsive personality disorder (OCD), paranoid, seasonal affective disorder, self-injury separation, sleep disorder, substance-induced mood disorder, Tourette syndrome, tic disorder, or Trichotillomania.

In another embodiment, the crystalline forms disclosed herein can be used to treat addiction in a subject in need thereof. The addiction can be drug addiction or alcohol addiction.

The drug addiction can be one or more of opioid addiction (i.e., opioid dependence) or stimulant addiction. The opioid can be one or more of fentanyl, morphine, oxymorphone, buprenorphine, hydromorphone, oxycodone, hydrocodone, or the like. The drug addiction can also be one or more of diamorphine (i.e., heroin), cocaine, nicotine, and amphetamine.

In one embodiment, the crystalline forms disclosed herein can be used to treat a disease or condition in a subject, wherein the subject has a tolerance to opioid medication, the subject has a history of opioid dependency or abuse, the subject is at risk of opioid dependency or abuse, or in circumstances wherein it is desirable that the risk of opioid dependence, opioid addiction, or symptoms of opioid withdrawal in the subject is minimized.

The crystalline forms disclosed herein can also be used to treat alcohol addiction, which can also be referred to as alcoholism. “Alcoholism” refers to an addictive disease or disorder characterized by an inability to control the intake of alcohol, i.e., a continued excessive or compulsive use of alcoholic drinks. Alcoholism may involve changes an individual's ability to metabolize alcohol as well. Diagnosis of alcoholism can be made by psychiatric examination.

In one aspect, the crystalline forms provided herein are useful in treatment of pain by acting as an agonist of the μ-opioid receptor.

In one embodiment of the methods described herein, the subject is human.

### Pharmaceutical Compositions

In an aspect, provided herein is a pharmaceutical composition comprising a crystalline form provided herein and a pharmaceutically acceptable carrier.

In an embodiment, the pharmaceutical composition comprises a crystalline form that is substantially free from other crystalline forms.

The pharmaceutical compositions can be formulated for oral, intravenous, intramuscular, subcutaneous or parenteral administration for the therapeutic or prophylactic treatment of pain, such as chronic pain and musculoskeletal pain.

The pharmaceutical preparations disclosed herein can be prepared in accordance with standard procedures and are administered at dosages that are selected to reduce, prevent or eliminate disease. See, for example, Remington's Pharmaceutical Sciences, Mack Publishing Company, Easton, PA and Goodman and Gilman's “The Pharmaceutical Basis of Therapeutics,” Pergamon Press, New York, N.Y., the contents of which are incorporated herein by reference, for a general description of the methods for administering various agents for human therapy.

The pharmaceutical compositions described herein can comprise a crystalline form disclosed herein in association with one or more nontoxic, pharmaceutically acceptable carriers and/or diluents and/or adjuvants and/or excipients.

For oral or parenteral administration, the crystalline form disclosed herein can be mixed with conventional pharmaceutical carriers and excipients and used in the form of tablets, capsules, elixirs, suspensions, syrups, wafers and the like. The compositions comprising a crystalline form disclosed herein can contain from about 0.1% to about 99% by weight of the active compound, such as from about 10% to about 30%.

For oral use, solid formulations such as tablets and capsules are useful. Sustained release or enterically coated preparations can also be devised. For pediatric and geriatric applications, one embodiment provides suspensions, syrups and chewable tablets. For oral administration, the pharmaceutical compositions are in the form of, for example, a tablet, capsule, suspension or liquid.

The pharmaceutical compositions can be made in the form of a dosage unit containing a therapeutically-effective amount of the active ingredient. Examples of such dosage units are tablets and capsules. For therapeutic purposes, the tablets and capsules which can contain, in addition to the active ingredient, conventional carriers such as binding agents, fillers, lubricants, disintegrants, or acceptable wetting agents. Oral liquid preparations generally are in the form of aqueous or oily solutions, suspensions, emulsions, syrups or elixirs.

The pharmaceutical compositions disclosed herein can be placed in a pharmaceutically acceptable carrier and are delivered to a recipient subject (e.g., a human) in accordance with known methods of drug delivery. In general, the methods of delivering the pharmaceutical compositions in vivo utilize art-recognized protocols for delivering the agent with the only substantial procedural modification being the substitution of a crystalline form of the present disclosure for the drugs in the art-recognized protocols.

### Administration/Dosage/Formulations

In another aspect, provided herein is a pharmaceutical composition comprising a crystalline form provided herein, together with a pharmaceutically acceptable carrier.

Actual dosage levels of the active ingredients in the pharmaceutical compositions discussed herein may be varied so as to obtain an amount of the active ingredient that is effective to achieve the desired therapeutic response for a particular patient, composition, and mode of administration, without being toxic to the patient.

In particular, the selected dosage level will depend upon a variety of factors including the activity of the particular compound employed, the time of administration, the rate of excretion of the compound, the duration of the treatment, other drugs, compounds or materials used in combination with the crystalline form, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could begin administration of the pharmaceutical composition to dose the disclosed crystalline form at levels lower than that required in order to achieve the desired therapeutic effect and gradually increase the dosage until the desired effect is achieved.

In particular embodiments, it is especially advantageous to formulate the crystalline form in dosage unit form for ease of administration and uniformity of dosage.

“Dosage unit form,” as used herein, refers to physically discrete units suited as unitary dosages for the patients to be treated; each unit containing a predetermined quantity of the disclosed compound calculated to produce the desired therapeutic effect in association with the required pharmaceutical vehicle. The dosage unit forms of the crystalline form disclosed herein are dictated by and directly dependent on (a) the unique characteristics of the disclosed compound and the particular therapeutic effect to be achieved, and (b) the limitations inherent in the art of compounding/formulating such a disclosed compound for the treatment of pain, a depressive disorder, or drug addiction in a patient.

In one embodiment, the crystalline form provided herein is formulated using one or more pharmaceutically acceptable excipients or carriers. In one embodiment, the pharmaceutical compositions comprise a therapeutically effective amount of the disclosed crystalline form and a pharmaceutically acceptable carrier.

In some embodiments, the dose of a disclosed compound is from about 1 mg to about 1,000 mg. In some embodiments, a dose of the disclosed compound used in compositions described herein is less than about 1,000 mg, or less than about 800 mg, or less than about 600 mg, or less than about 500 mg, or less than about 300 mg, or less than about 200 mg, or less than about 100 mg, or less than about 50 mg, or less than about 20 mg, or less than about 10 mg. For example, a dose is about 10 mg, 20 mg, 25 mg, 30 mg, 40 mg, 50 mg, 60 mg, 70 mg, 80 mg, 90 mg, 100 mg, 120 mg, 140 mg, 160 mg, 180 mg, 200 mg, 220 mg, 240, 260 mg, 280 mg, 300 mg, 350 mg, 400 mg, 450 mg, 500 mg, 550 mg, or about 600 mg.

Routes of administration of any of the compositions disclosed herein include oral, nasal, rectal, intravaginal, parenteral, buccal, sublingual or topical. The compound for use provided herein may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration. In one embodiment, the preferred route of administration is oral.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example, an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For parenteral administration, the disclosed compound may be formulated for injection or infusion, for example, intravenous, intramuscular or subcutaneous injection or infusion, or for administration in a bolus dose or continuous infusion. Suspensions, solutions or emulsions in an oily or aqueous vehicle, optionally containing other formulatory agents such as suspending, stabilizing or dispersing agents may be used.

Those skilled in the art will recognize, or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings of the present disclosure as set forth.

### EXAMPLES

The disclosure is further illustrated by the following examples, which should not be construed as further limiting. The practice of the present disclosure will employ, unless otherwise indicated, conventional techniques of organic synthesis, cell biology, cell culture, and molecular biology, which are within the skill of the art.

### Analytical Methods

Unless otherwise indicated, PXRD was performed on a Rigaku MiniFlex II diffractometer (Rigaku/MSC, Woodlands, Tex.) in reflection mode using collimated Cu Kα radiation operating at 30 kV/15 mA. Scans were run from 2-40 degrees 2-theta with a step size of 0.02 degrees and a scan time of 7.6 degrees/minute. Data were viewed with Bruker-AXS DIFFRAC.EVA version 3.0.

DSC was conducted on a TA Instruments DSC Q2500 with Tzero aluminum crimped pans. DSC analysis was performed by ramping 10 degrees C. per minute from 0 degrees to 300 degrees C. Data analysis was performed using TA Instruments Trios v4.3.1.39215.

### Example 1

### Synthesis of (1R,5S,9r-3-benzyl-9-(3-iodophenyl)-9-ethoxy-3-azabicyclo[3.3.1]-nonane

The following synthesis is disclosed in PCT Publication No. WO 2019/152946 and U.S. patent application Ser. No. 16/267,025, the entire contents of which are incorporated herein by reference.

Synthesis of 3-benzyl-1,5,3-dioxazepane

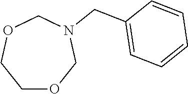 [SMILES: c1ccc(CN2COCCOC2)cc1]

A mixture of benzylamine (88.0 g, 0.82 mol), paraformaldehyde (61.6 g, 2.05 mol) and ethylene glycol (55.0 mL, 0.98 mol) in toluene (350 mL) was heated at reflux for 2 hours with a Dean Stark trap. The mixture was cooled, and then partitioned between ethyl acetate and brine. The aqueous phase was extracted with ethyl acetate and the combined organic phases were dried over MgSO₄, filtered and concentrated under reduced pressure to give 3-benzyl-1,5,3-dioxazepane (100.0 g, 68% yield); ¹H NMR (300 MHz, CDCl₃): 7.20-7.43 (m, 5H), 4.48 (s, 4H), 4.02 (s, 2H), 3.89 (s, 4H).

Synthesis of (1R,5S)-3-benzyl-3-azabicyclo[3.3.1]nonan-9-one

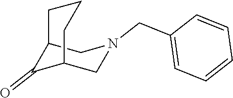 [SMILES: O=C1C2CCCC1CN(Cc1ccccc1)C2]

To a solution of 3-benzyl-1,5,3-dioxazepane (100 g, 0.52 mol) in methanol (600 mL) at 5° C. was added acetyl chloride (111 mL, 1.55 mol) over 10 minutes. After stirring for 5 minutes, a solution of cyclohexanone (51 g, 0.52 mol) in methanol (60 mL) was added. The mixture was stirred for 10 minutes, then allowed to warm to room temperature and stirred for 4 hours. The mixture was poured into concentrated aqueous ammonia and extracted with ethyl acetate (×2). The combined organic phases were washed with brine, dried over MgSO₄, filtered and concentrated under reduced pressure. The residue was dissolved in 4.4 M aqueous hydrochloric acid (500 mL) and heated at 50° C. for 3 hours. The reaction mixture was cooled and poured into ice/concentrated aqueous ammonia. The mixture was extracted with ethyl acetate (×2) and the combined organic phases were dried over MgSO₄, filtered and concentrated under reduced pressure. The residue was purified by silica chromatography, eluting with 40-100% dichloromethane in heptane, to give (1R,5S)-3-benzyl-3-azabicyclo[3.3.1]nonan-9-one (95 g, 50% yield); [M+H]⁺ 230.13.

Synthesis of (1R, 5S,9s)-3-benzyl-9-(3-iodophenyl)-3-azabicyclo[3.3.1]nonan-9-ol and (1R, 5S,9r-3-benzyl-9-(3-iodophenyl)-3-azabicyclo[3.3.1]nonan-9-ol

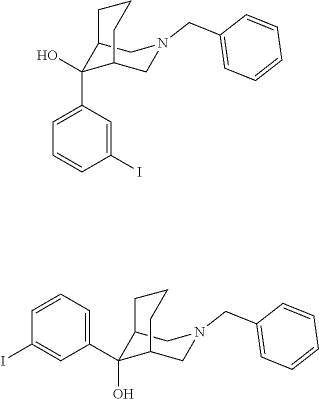 [SMILES: OC1(c2cccc(I)c2)C2CCCC1CN(Cc1ccccc1)C2 ; OC1(c2cccc(I)c2)C2CCCC1CN(Cc1ccccc1)C2]

n-Butyl lithium (1.5 M in hexanes, 49.9 mL, 74.8 mmol) was added drop wise to a solution of 1,3-diiodobenzene (41.2 g, 124.7 mmol) in diethyl ether (300 mL) at −78° C. under argon. After 40 minutes at −78° C., a solution of (1R,5S)-3-benzyl-3-azabicyclo[3.3.1]nonan-9-one (14.3 g, 62.4 mmol) in diethyl ether (50 mL) was added drop wise. The reaction mixture was allowed to warm to room temperature over 1 hour. The reaction was quenched with water and then poured into a mixture of 2 M aqueous hydrochloric acid and diethyl ether. The phases were separated and the organic phase washed with 2 M aqueous hydrochloric acid. The acidic phases were combined, basified with concentrated ammonium hydroxide and extracted with dichloromethane (×3). The combined dichloromethane layers were washed with brine, dried over MgSO₄, filtered and concentrated under reduced pressure to give a 1:2 ratio of (1R,5S,9r-3-benzyl-9-(3-iodophenyl)-3-azabicyclo[3.3.1]nonan-9-ol, and (1R,5S,9s)-3-benzyl-9-(3-iodophenyI)-3-azabicyclo[3.3.1]nonan-9-ol (24.3 g, 90% yield); [M+H]⁺ 434.28.

Epimerisation of (1R, 5S,95)-3-benzyl-9-(3-iodophenyl)-3-azabicyclo[3.3.1]nonan-9-ol to (1R, 5S,9r-3-benzyl-9-(3-iodophenyl)-3-azabicyclo[3.3.1]nonan-9-ol

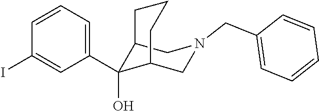 [SMILES: OC1(c2cccc(I)c2)C2CCCC1CN(Cc1ccccc1)C2]

To a stirred solution of (1R,5S,9s)-3-benzyl-9-(3-iodophenyI)-3-azabicyclo[3.3.1]-nonan-9-ol and (1R,5S,9r-3-benzyl-9-(3-iodophenyl)-3-azabicyclo[3.3.1]nonan-9-ol (24.1 g, 55.6 mmol), in a 2:1 ratio, in 1,4-dioxane (150 mL) was added water (80 mL) followed by 6 M aqueous sulfuric acid (300 mL) and the mixture was stirred at 100° C. for 16 hours. Further 6 M aqueous sulfuric acid (100 mL) was added and the reaction stirred for 1 hour at 100° C. The reaction was poured onto ice/concentrated aqueous ammonia and extracted with dichloromethane (×3). The combined organic phases were washed with brine, dried over MgSO₄, filtered and concentrated under reduced pressure to give a 9:2 ratio of (1R,5S,9r)-3-benzyl-9-(3-iodophenyl)-3-azabicyclo[3.3.1]nonan-9-ol:(1R,5S,9s)-3-benzyl-9-(3-iodophenyl)-3-azabicyclo[3.3.1]nonan-9-ol (26.8 g, 100% yield). Purification of 16.5 g of this material by silica chromatography, eluting with 0-10% ethyl acetate in toluene:heptane (1:1) gave (1R,5S,9r-3-benzyl-9-(3-iodophenyl)-3-azabicyclo[3.3.1]nonan-9-ol (9.3 g); [M+H]⁺ 434.54; and (1R,5S,9s)-3-benzyl-9-(3-iodophenyl)-3-azabicyclo[3.3.1]nonan-9-ol (1.9 g); [M+H]⁺ 434.29.

Synthesis of (1R, 5S,9r)-3-benzyl-9-(3-iodophenyl)-9-ethoxy-3-azabicyclo[3.3.1]nonane

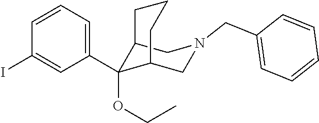 [SMILES: CCOC1(c2cccc(I)c2)C2CCCC1CN(Cc1ccccc1)C2]

To an ice cold solution of (1R,5S,9r-3-benzyl-9-(3-iodophenyl)-3-azabicyclo[3.3.1]-nonan-9-ol (3.99 g, 9.21 mmol) in dimethyl sulfoxide (20 mL) was added sodium hydride (60% dispersion in oil, 0.66 g, 16.58 mmol) portion wise. After 10 minutes, iodoethane (0.86 mL, 13.81 mmol) was added. The reaction mixture was allowed to warm to room temperature, and stirred for 1 hour. A further portion of sodium hydride (60% dispersion in oil, 110 mg, 2.75 mmol) was added, followed by iodomethane (0.17 mL, 2.75 mmol) and the reaction mixture was stirred for 1 hour. The reaction was quenched by pouring into ice/water and extracted with ethyl acetate (×3). The combined organic phases were washed with brine, dried over MgSO₄, filtered and concentrated under reduced pressure. The crude material was purified by silica chromatography, eluting with 5-7% ethyl acetate in heptane, to give (1R,5S,9r-3-benzyl-9-(3-iodophenyl)-9-ethoxy-3-azabicyclo[3.3.1]nonane (3.24 g, 78% yield); [M+H]⁺ 448.30.

Synthesis of 3-((1R,5S,9r)-3-benzyl-9-ethoxy-3-azabicyclo[3.3.1]nonan-9-yl)-benzonitrile

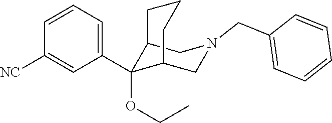 [SMILES: CCOC1(c2cccc(C#N)c2)C2CCCC1CN(Cc1ccccc1)C2]

To a solution of (1R,5S,9r-3-benzyl-9-(3-iodophenyl)-9-ethoxy-3-azabicyclo[3.3.1]-nonane (3.24 g, 7.20 mmol) in degassed N,N-dimethylformamide (35 mL) was added tris-(dibenzylideneacetone)dipalladium (0) (0.66 g, 0.72 mmol) and 1,1′- bis(diphenylphosphino)-ferrocene (0.80 g, 1.50 mmol). After heating to 50° C., zinc cyanide (0.51 g, 4.34 mmol) was added and the reaction mixture heated at 110° C. for 2 hours. The reaction mixture was cooled to room temperature, quenched with sodium hydrogen carbonate solution, diluted with ethyl acetate and filtered through a pad of Celite. The product was extracted with ethyl acetate (×3). The combined organic phases were washed with water (×2), brine (×1), dried over MgSO₄, filtered and concentrated under reduced pressure. The crude material was purified by silica chromatography, eluting with 0-20% ethyl acetate in heptane, to give 3-((1R,5S,9r-3-benzyl-9-ethoxy-3-azabicyclo[3.3.1]nonan-9-yl)-benzonitrile (1.89 g); [M+H]⁺ 347.36.

Synthesis of 3-((1R,5S,9r)-3-benzyl-9-ethoxy-3-azabicyclo[3.3.1]nonan-9-yl)-benzamide

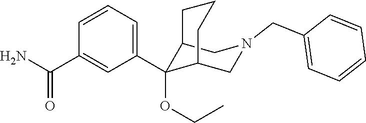 [SMILES: CCOC1(c2cccc(C(N)=O)c2)C2CCCC1CN(Cc1ccccc1)C2]

To 3-((1R,5S,9r-3-benzyl-9-ethoxy-3-azabicyclo[3.3.1]nonan-9-yl)-benzonitrile (1.89 g, 5.50 mmol) was added tert-butanol (55 mL), followed by potassium hydroxide (1.53 g, 27.3 mmol) and the reaction was heated at reflux for 2 hours. The mixture was cooled to room temperature, diluted with water and extracted with ethyl acetate (×3). The combined organic phases were washed with water (×2), then brine (×1), dried over MgSO₄, filtered and concentrated under reduced pressure. The crude material was purified by silica chromatography, eluting with 50-66% ethyl acetate in heptane, to give 3-((1 R,5S,9r)-3-benzyl-9-ethoxy-3-azabicyclo[3.3.1]nonan-9-yl)-benzamide (1.03 g, 39% yield over 2 steps); [M+H]⁺ 365.38.

Synthesis of 3-((1R,5S,9r)-9-ethoxy-3-azabicyclo[3.3.1]nonan-9-yl)-benzamide

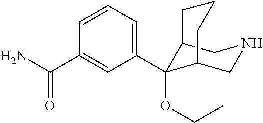 [SMILES: CCOC1(c2cccc(C(N)=O)c2)C2CCCC1CNC2]

A mixture of 3-((1R,5S,9r-3-benzyl-9-ethoxy-3-azabicyclo[3.3.1]nonan-9-yl)-benzamide (980 mg, 2.70 mmol), 20% palladium hydroxide on carbon (98 mg), and ammonium formate (1.70 g, 27.00 mmol) in methanol (25 mL) was heated at reflux for 30 minutes. Further ammonium formate (1.70 g, 27.00 mmol) and 20% palladium hydroxide on carbon (49 mg) were added and the mixture heated at reflux for 30 minutes. The mixture was cooled to room temperature and filtered through a pad of Celite washing thoroughly with methanol. The filtrate was concentrated under reduced pressure. The residue was taken up in dichloromethane and concentrated aqueous ammonia/water (1:1) and extracted with dichloromethane (×3). The combined organic phases were washed with brine, dried over MgSO₄, filtered and concentrated under reduced pressure. The residue was purified by reverse phase chromatography (C18) to give 3-((1R,5S,9r-9-ethoxy-3-azabicyclo[3.3.1]nonan-9-yl)-benzamide (273 mg, 37% yield); [M+H]⁺ 275.18.

### Example 2

### 3-((1R,5S,9r-9-ethoxy-3-azabicyclo-[3.3.1]nonan-9-yl)-benzamide Free Base Form A

The free base Form A was obtained according to the following method: 3-((1R,5S,9r)-9-ethoxy-3-azabicyclo-[3.3.1]nonan-9-yl)-benzamide hydrochloride was suspended in 25 volumes of acetone and then neutralized with 1.1 molar equivalents of 1 N sodium hydroxide with mixing. The solvent was stripped off to achieve an oil. The oil was suspended in 20 volumes tert-butyl methylether followed by the addition of 60 volumes of diethyl ether to precipitate a flocculated amorphous solid. The sample was evaporated to about half the initial volume until amorphous solids began to stick to the sides of the vessel. The sample was sonicated and mixed until solids became dislodged from sides of vial and birefringent needles began to form (as observed by polarized light microscope). The sample was allowed to stir overnight at room temperature or until sample was completely converted to birefringent needles.

The PXRD diffractogram for the free base Form A provided herein is shown in FIG. 1. Table 1 also shows PXRD data for a sample of 3-((1 R,5S,9r)-9-ethoxy-3-azabicyclo-[3.3.1]-nonan-9-yl)-benzamide free base Form A.

TABLE 1

| Peak Number | Angle (degrees 2θ) |
|---|---|
| 1 | 6.7 |
| 2 | 9.9 |
| 3 | 11.4 |
| 4 | 12.2 |
| 5 | 12.6 |
| 6 | 13.6 |
| 7 | 14.3 |
| 8 | 14.5 |
| 9 | 15.1 |
| 10 | 15.6 |
| 11 | 17.2 |
| 12 | 18.4 |
| 13 | 19.2 |
| 14 | 20.3 |
| 15 | 20.6 |
| 16 | 22.6 |
| 17 | 23.1 |
| 18 | 24.2 |
| 19 | 25.6 |
| 20 | 26.2 |
| 21 | 27.0 |
| 22 | 27.6 |
| 23 | 28.4 |
| 24 | 31.5 |

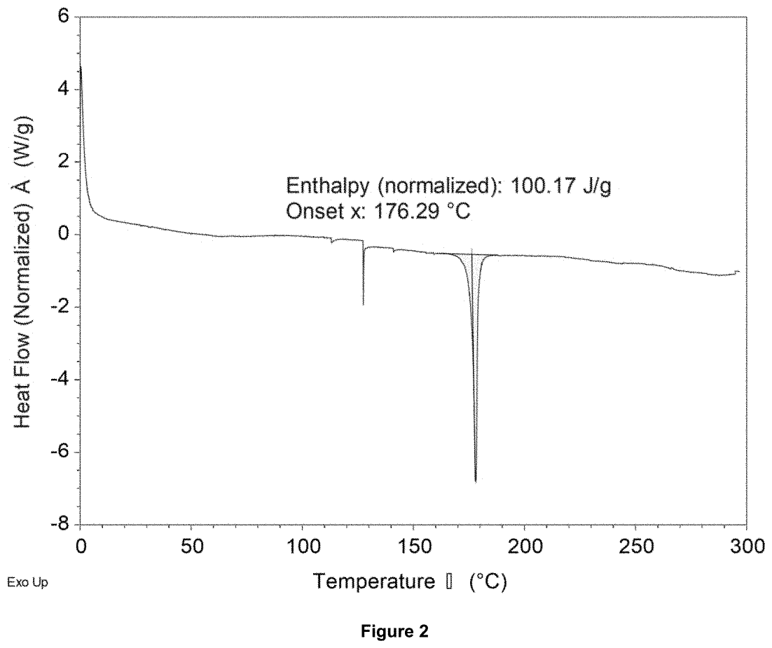

DSC analysis of the free base Form A was performed by ramping 10 degrees C. per minute from 0 degrees to 300 degrees C. The DSC thermogram is depicted in FIG. 2. An endotherm is observed with an onset temperature of 176 degrees C.

### Example 3

### 3-((1R,5S,9r-9-ethoxy-3-azabicyclo-[3.3.1]nonan-9-yl)-benzamide hydrochloride Form A

The hydrochloride Form A was obtained according to the following method:

To a stirred solution of 4 M HCl in isopropyl alcohol (90 mL, 360 mmol) was added tert-butyl 9-(3-carbamoylphenyl)-9-ethoxy-3-azabicyclo[3.3.1]nonane-3-carboxylate (8 g, 20.6 mmol, 1 equiv.) in portions at room temperature under air atmosphere. The resulting mixture was stirred for 1 hour at room temperature under nitrogen atmosphere. The reaction was monitored by LCMS. The resulting mixture was concentrated under reduced pressure. The precipitated solids were collected by filtration and washed with isopropyl alcohol (3×15 mL). The resulting solid was collected, dried under vacuum and freeze-dried two times to remove the residual isopropyl alcohol to afford the product.

The PXRD diffractogram for the hydrochloride Form A provided herein is shown in FIG. 3. Table 2 also shows PXRD data for a sample of 3-((1R,5S,9r-9-ethoxy-3-azabicyclo-[3.3.1]nonan-9-yl)-benzamide hydrochloride Form A.

TABLE 2

| Peak Number | Angle (degrees 2θ) |
|---|---|
| 1 | 10.9 |
| 2 | 12.2 |
| 3 | 12.9 |
| 4 | 13.7 |
| 5 | 15.5 |
| 6 | 17.2 |
| 7 | 18.4 |
| 8 | 19.9 |
| 9 | 21.0 |
| 10 | 22.1 |
| 11 | 24.5 |
| 12 | 26.3 |
| 13 | 26.7 |
| 14 | 27.2 |
| 15 | 27.8 |
| 16 | 29.1 |
| 17 | 29.8 |
| 18 | 30.7 |
| 19 | 31.5 |

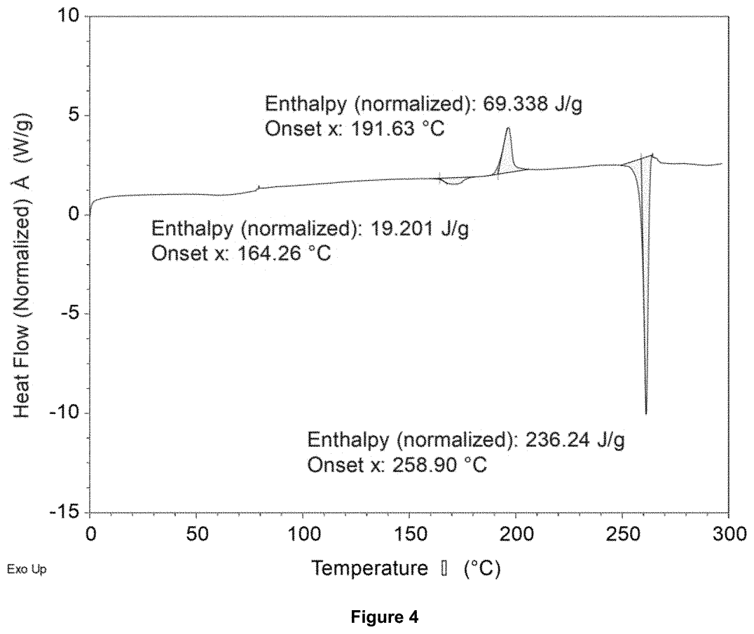

DSC analysis of the hydrochloride Form A was performed by ramping 10 degrees C. per minute from 0 degrees to 300 degrees C. The DSC thermogram is depicted in FIG. 4. A broad endotherm with an onset temperature at 164 degrees C. is followed by an exotherm with an onset temperature at 192 degrees C. The sharp endotherm with an onset at 259 degrees C. is likely from the melt of Form B recrystallized from Form A converted during the run.

### Example 4

### 3-((1R,5S,9r-9-ethoxy-3-azabicyclo-[3.3.1]nonan-9-yl)-benzamide hydrochloride Form B

3-((1R,5S,9r-9-ethoxy-3-azabicyclo-[3.3.1]nonan-9-yl)-benzamide free base was dissolved in 1 N HCl. Water was removed by evaporation to precipitate hydrochloride Form B as a crystalline solid. The solids were filtered and dried.

The PXRD diffractogram for the hydrochloride Form B provided herein is shown in

FIG. 5. Table 3 also shows PXRD data for a sample of 3-((1R,5S,9r-9-ethoxy-3-azabicyclo-[3.3.1]nonan-9-yl)-benzamide hydrochloride Form B.

TABLE 3

| Peak Number | Angle (degrees 2θ) |
|---|---|
| 1 | 9.0 |
| 2 | 10.0 |
| 3 | 10.8 |
| 4 | 12.4 |
| 5 | 12.8 |
| 6 | 14.7 |
| 7 | 15.4 |
| 8 | 16.6 |
| 9 | 17.6 |
| 10 | 18.1 |
| 11 | 18.5 |
| 12 | 19.3 |
| 13 | 20.1 |
| 14 | 20.3 |
| 15 | 21.7 |
| 16 | 22.0 |
| 17 | 22.3 |
| 18 | 23.2 |
| 19 | 24.4 |
| 20 | 25.1 |
| 21 | 25.3 |
| 22 | 25.9 |
| 23 | 26.2 |
| 24 | 27.5 |
| 25 | 28.5 |
| 26 | 29.8 |
| 27 | 30.6 |
| 28 | 32.0 |
| 29 | 33.1 |
| 30 | 33.7 |
| 31 | 34.5 |
| 32 | 35.1 |
| 33 | 35.5 |
| 34 | 39.2 |

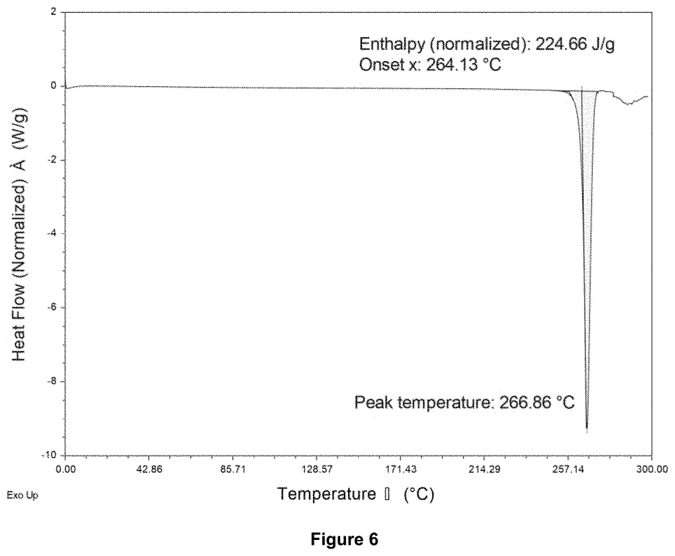

DSC analysis of the hydrochloride Form B was performed by ramping 10 degrees C. per minute from 0 degrees to 300 degrees C. The DSC thermogram is depicted in FIG. 6. An endotherm is observed with an onset temperature of 264 degrees C. and a peak temperature of 267 degrees C.

### Example 5

### 3-((1R,5S,9r-9-ethoxy-3-azabicyclo-[3.3.1]nonan-9-yl)-benzamide hydrochloride Form D

3-((1R,5S,9r-9-ethoxy-3-azabicyclo[3.3.1]nonan-9-yl)-benzamide hydrochloride Form B was slurried with heated ethanol to form a saturated solution. The slurry was filtered with a 0.2 μM filter to remove residual solids. Evaporation of ethanol at ambient conditions resulted in crystallization of the hydrochloride Form D.

PXRD was performed on a Rigaku MiniFlex 6G diffractometer (Rigaku/MSC, Woodlands, Tex.) in reflection mode using collimated Cu Kα radiation operating at 30 kV/15 mA. Scans were run from 2-40 degrees 2-theta with a step size of 0.02 degrees and a scan time of 7.6 degrees/min. Data were viewed with Bruker-AXS DIFFRAC.EVA version 3.0.

The PXRD diffractogram for the hydrochloride Form D provided herein is shown in FIG. 7. Table 4 also shows PXRD data for a sample of 3-((1R,5S,9r-9-ethoxy-3-azabicyclo-[3.3.1]nonan-9-yl)-benzamide hydrochloride Form D.

TABLE 4

| Peak Number | Angle (degrees 2θ) |
|---|---|
| 1 | 9.1 |
| 2 | 9.7 |
| 3 | 10.4 |
| 4 | 11.6 |
| 5 | 11.9 |
| 6 | 13.8 |
| 7 | 15.8 |
| 8 | 17.1 |
| 9 | 17.6 |
| 10 | 18.3 |
| 11 | 19.0 |
| 12 | 19.6 |
| 13 | 21.0 |
| 14 | 21.6 |
| 15 | 21.7 |
| 16 | 22.5 |
| 17 | 23.9 |
| 18 | 24.1 |
| 19 | 25.1 |
| 20 | 26.1 |
| 21 | 27.6 |
| 22 | 29.2 |
| 23 | 29.5 |
| 24 | 30.4 |
| 25 | 30.7 |
| 26 | 31.7 |
| 27 | 32.1 |
| 28 | 32.8 |
| 29 | 33.3 |
| 30 | 33.7 |
| 31 | 34.2 |
| 32 | 34.9 |
| 33 | 36.6 |
| 34 | 38.2 |

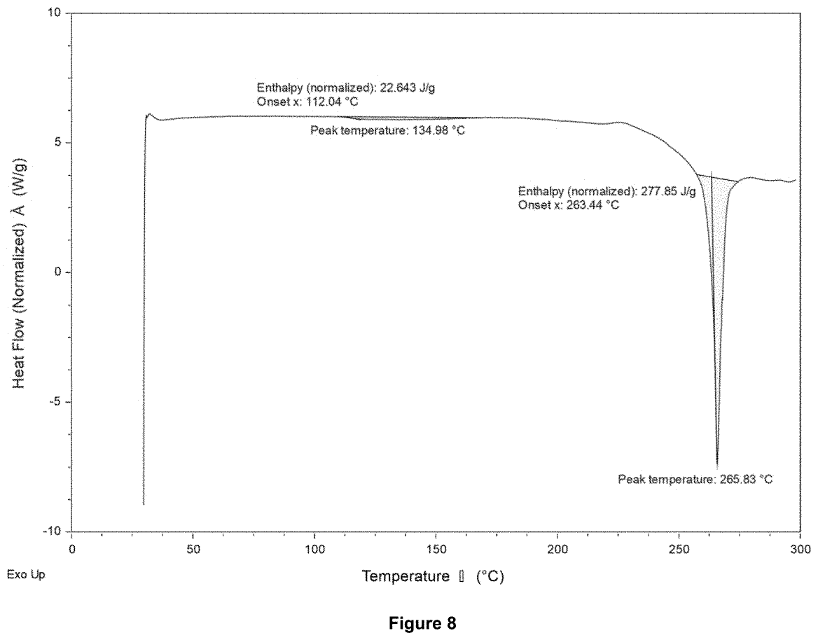

DSC analysis of the hydrochloride Form D was performed by ramping 10 degrees C. per minute from 0 degrees to 300 degrees C. The DSC thermogram is depicted in FIG. 8. An endotherm is observed with an onset temperature at 263 degrees C. is followed by a melt with a peak temperature at 266 degrees C.

### Example 6

### Dynamic Vapor Sorption (DVS) of 3-((1R,5S,9r-9-ethoxy-3-azabicyclo-[3.3.1]nonan-9-yl)-benzamide hydrochloride Form D

DVS analysis was conducted using a TA Instruments TGA Q5000 SA. DVS was run at 25 degrees C. The sample was pre-dried at 25 degrees C. and 0% relative humidity (% RH) until the dm/dt was 0.01% for up to 300 minutes. Adsorption and desorption isotherms were measured by ramping the humidity from 0% RH to 90% RH to 0% RH at 10% RH step intervals with a stability criterion of dm/dt=0.01% or 300 minutes unless otherwise noted. Data analysis was performed using Universal Analysis 2000 Version 4.5 A.

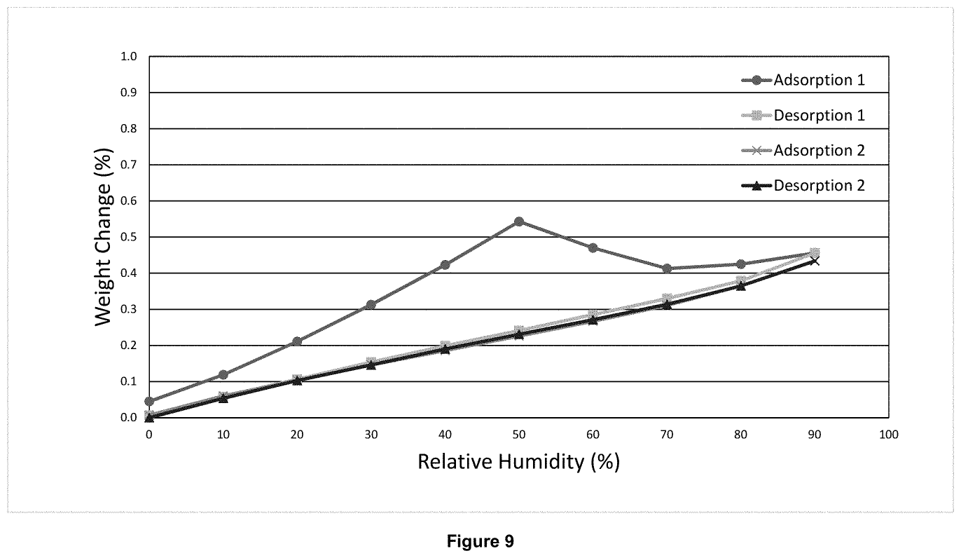

DVS analysis of hydrochloride Form D showed approximately 0.5% water up to 50% RH, followed by a decrease in weight at 60% RH, indicating a change in the material. A change in the profile was observed in the second cycle, with a decreased amount of water gained and no inflection at 50% RH as in the first cycle. FIG. 9 shows the DVS isotherm plot, normalized to minimum weight.

The measured PXRD pattern for the material following two cycle DVS showed the pattern has changed from the pre-DVS starting material, indicating a form change. The post-DVS pattern is consistent with hydrochloride Form B.

Based on this experiment, Form B appears to be the most stable HCl salt solid form.

Various modifications of the disclosure, in addition to those described herein, will be apparent to those skilled in the art from the foregoing description. Such modifications are also intended to fall within the scope of the appended claims. Each reference, including without limitation all patent, patent applications, and publications, cited in the present application is incorporated herein by reference in its entirety.

1. A crystalline form of 3-((1R,5S,9r)-9-ethoxy-3-azabicyclo[3.3.1]nonan-9-yl)-benzamide hydrochloride Form B.

2. The crystalline form of claim 1, wherein the crystalline form is characterized by an PXRD diffractogram having peaks expressed in degrees-2-theta at angles (±0.2 degrees) of 9.0, 10.8, and 15.4.

3. The crystalline form of claim 1, wherein the crystalline form is characterized by an PXRD diffractogram having peaks expressed in degrees-2-theta at angles (±0.2 degrees) of 9.0, 10.8, 12.4, and 14.7.

4. The crystalline form of claim 1, wherein the crystalline form is characterized by an PXRD diffractogram having peaks expressed in degrees-2-theta at angles (±0.2 degrees) of 9.0, 10.8, 12.4, 14.7, 15.4, 18.5 and 20.1.

5. The crystalline form of claim 1, wherein the crystalline form is characterized by an PXRD diffractogram having peaks expressed in degrees-2-theta at angles (±0.2 degrees) of 9.0, 10.0, 10.8, 12.4, 14.7, 15.4, 18.1, 18.5, 20.1, and 22.3.

6. The crystalline form of claim 1, wherein the crystalline form has the PXRD diffractogram substantially as depicted in FIG. 5.

7. The crystalline form of claim 1 having a DSC thermogram characterized by an endotherm with an onset temperature of 264 degrees C.

8. The crystalline form of claim 1 having a DSC thermogram characterized by an endothermic peak at about 267 degrees C.

9. A pharmaceutical composition comprising the crystalline form of claim 1 and a pharmaceutically acceptable carrier.

10. A method of treating pain in a subject in need thereof comprising administering to the subject the crystalline form of claim 1.

11. The method of claim 10, wherein the pain is inflammatory pain, thermal pain, acute pain, chronic pain, traumatic pain, chemical pain, ischemic pain, centrally mediated pain, peripherally mediated pain, prickling pain, visceral pain, progressive disease pain, musculoskeletal pain and neuropathic pain.

12. The method of claim 11, wherein the pain is inflammatory pain, thermal pain, acute pain, chronic pain, musculoskeletal pain, and neuropathic pain.

13. The method of claim 11, wherein the pain is chronic pain.

14. The method of claim 11, wherein the pain is musculoskeletal pain.
